(19) United States
(12) Patent Application Publication
(10) Pub. No.: US 2017/0197948 A1
(43) Pub. Date: Jul. 13, 2017

(54) METHOD FOR PRODUCING CARBOXYLIC ANHYDRIDE

(71) Applicant: JX NIPPON OIL & ENERGY CORPORATION, Tokyo (JP)

(72) Inventors: Rieko FUJISHIRO, Tokyo (JP); Ayumu KOMATA, Tokyo (JP); Takeshi KOIKE, Tokyo (JP); Shinichi KOMATSU, Tokyo (JP)

(73) Assignee: JX NIPPON OIL & ENERGY CORPORATION, Tokyo (JP)

(21) Appl. No.: 15/312,809

(22) PCT Filed: May 13, 2015
(86) PCT No.: PCT/JP2015/063723

§ 371 Date: Nov. 21, 2016

(30) Foreign Application Priority Data
May 21, 2014 (JP) 2014-105521

(51) Int. Cl. C07D 407/08

(52) U.S. Cl. CPC C07D 407/08

(57) ABSTRACT

Method for producing carboxylic anhydride, the method includes heating raw material compound represented by the following general formula (1):

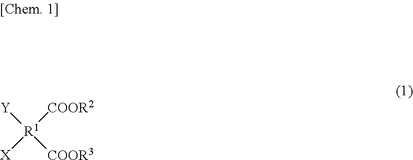

[in the formula (1), R¹ is tetravalent organic group having at least two carbon atoms adjacent to each other, and groups represented by the formulae: —COOR² and —COOR³ are bonded to one and the other of the two adjacent carbon atoms, respectively, R² and R³ each represent hydrogen atom or the like, X represents hydrogen atom or the like, and Y represents hydrogen atom or the like] in carboxylic acid having 1 to 5 carbon atoms with catalyst being used, to thereby obtain the carboxylic anhydride, wherein the catalyst is homogeneous acid catalyst having acid dissociation constant (pKa) of −6.5 or lower and boiling point of 100° C. or higher, the acid dissociation constant (pKa) being determined by quantum chemistry calculation based on density functional method.

### TECHNICAL FIELD

The present invention relates to a method for producing a carboxylic anhydride.

### BACKGROUND ART

Carboxylic anhydrides have been used as raw materials for polyimides, polyesters, polyamides, and the like, curing agents for thermosetting resins, and the like. Various methods have been known as methods for producing such carboxylic anhydrides, and, for example, Japanese Unexamined Patent Application Publication No Hei 5-140141 (PTL 1) discloses a method for producing a carboxylic anhydride, the method comprising heating a carboxylic acid or carboxylic acid ester represented by the following general formula (A) in a lower carboxylic acid with a catalyst being used:

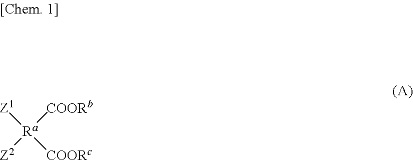

[in the general formula (A) R^{a} is a divalent to tetravalent organic group, Z¹ is a hydrogen atom, an alkyl group having 1 to 6 carbon atoms, or a —COOR^{d} group, and Z² is a hydrogen atom, an alkyl group having 1 to 6 carbon atoms, or a —COOR^{e} group, provided that Rb to R^{e}, which may be the same or different, each represent a hydrogen atom or an alkyl group having 1 to 6 carbon atoms].

### CITATION LIST

### Patent Literature

[PTL 1] Japanese Unexamined Patent Application Publication No. Hei 5-140141

### SUMMARY OF INVENTION

### Technical Problem

However, in a case where a so-called homogeneous catalyst (for example, toluenesulfonic acid or the like) described in PTL 1 is used as the catalyst in a method as described in PTL 1, a carboxylic anhydride in which color development is sufficiently suppressed cannot always be obtained. Note that it is possible to more efficiently obtain a product in a case where a homogeneous catalyst is used than in a case where a heterogeneous catalyst is used, because the step of separating the product from the catalyst after production of the product and the like can be omitted. For this reason, there has been a demand for development of a method for producing a carboxylic anhydride, the method using a homogeneous catalyst to obtain the product more efficiently but being capable of producing a carboxylic anhydride in which color development is sufficiently suppressed.

The present invention has been made in view of the problems of the above-described conventional technique, and an object of the present invention is to provide a method for producing a carboxylic anhydride, the method being capable of efficiently producing a carboxylic anhydride in which color development is sufficiently suppressed from the original color of crystals, although a homogeneous catalyst is used.

### Solution to Problem

The present inventors have conducted intensive study to achieve the above-described object, and consequently have found that, by a method for producing a carboxylic anhydride, the method comprising heating a raw material compound represented by the following general formula (1) in a carboxylic acid having 1 to 5 carbon atoms with a catalyst being used to thereby obtain the carboxylic anhydride, wherein the catalyst is a homogeneous acid catalyst having an acid dissociation constant (pKa) of −6.5 or lower and a boiling point of 100° C. or higher, the acid dissociation constant (pKa) being determined by a quantum chemistry calculation based on a density functional method (DFT method), it is possible to efficiently produce a carboxylic anhydride in which color development is sufficiently suppressed from the original color of crystals, although a homogeneous catalyst is used. This finding has led to the completion of the present invention.

Specifically, a method for producing a carboxylic anhydride of the present invention comprises

heating a raw material compound represented by the following general formula (1):

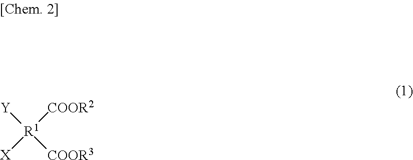

[in the formula (1),

R¹ is a tetravalent organic group having at least two carbon atoms adjacent to each other, and groups represented by the formulae: —COOR² and —COOR³ are bonded to one and the other of the two adjacent carbon atoms, respectively,

R² and R³, which may be the same or different, each represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms,

X represents one selected from the group consisting of a hydrogen atom, alkyl groups having 1 to 10 carbon atoms, alkenyl groups having 2 to 10 carbon atoms, and groups represented by the formula: —COOR⁴ (R⁴ has the same meaning as that of R², and may be the same as R² or different from R²), and

Y represents one selected from the group consisting of a hydrogen atom, alkyl groups having 1 to 10 carbon atoms, alkenyl groups having 2 to 10 carbon atoms, and groups represented by the formula: —COOR⁵ (R⁵ has the same meaning as that of R², and may be the same as R² or different from R²)]

in a carboxylic acid having 1 to 5 carbon atoms with a catalyst being used, to thereby obtain the carboxylic anhydride, wherein

the catalyst is a homogeneous acid catalyst having an acid dissociation constant (pKa) of −6.5 or lower and a boiling point of 100° C. or higher, the acid dissociation constant (pKa) being determined by a quantum chemistry calculation based on a density functional method.

In the above-described method for producing a carboxylic anhydride of the present invention, the homogeneous acid catalyst is preferably at least one selected from the group consisting of trifluoromethanesulfonic acid, tetrafluoroethanesulfonic acid, pentafluoroethanesulfonic acid, heptafluoropropanesulfonic acid, heptafluoroisopropanesulfonic acid, nonafluorobutanesulfonic acid, heptafluorodecanesulfonic acid, bis(nonafluorobutanesulfonyl)imide, N,N-bis(trifluoromethanesulfonyl)imide, and chlorodifluoroacetic acid.

In addition, in the above-described method for producing a carboxylic anhydride of the present invention, the raw material compound is preferably a spiro compound represented by the following general formula (2):

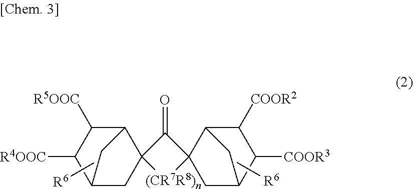

[in the formula (2), R², R³, R⁴, and R⁵ have the same meanings as those of R², R³, R⁴, and R⁵ described for the general formula (1), R⁶s, R⁷, and R⁸, which may be the same or different, each represent one selected from the group consisting of a hydrogen atom, alkyl groups having 1 to 10 carbon atoms, and a fluorine atom, and n represents an integer of 0 to 12].

### Advantageous Effects of Invention

According to the present invention, it is possible to provide a method for producing a carboxylic anhydride, the method being capable of efficiently producing a carboxylic anhydride in which color development is sufficiently suppressed from the original color of crystals, although a homogeneous catalyst is used.

### BRIEF DESCRIPTION OF DRAWINGS

FIG. 1 shows a reaction scheme of an acid dissociation reaction (dissociation reaction of a proton) of an acid catalyst.

### DESCRIPTION OF EMBODIMENTS

Hereinafter, the present invention will be described in detail based on preferred embodiments thereof.

A method for producing a carboxylic anhydride of the present invention comprises

heating a raw material compound represented by the following general formula (1):

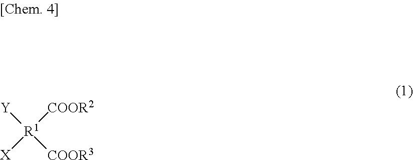

[in the formula (1),

R¹ is a tetravalent organic group having at least two carbon atoms adjacent to each other, and groups represented by the formulae: —COOR² and —COOR³ are bonded to one and the other of the two adjacent carbon atoms, respectively,

R² and R³, which may be the same or different, each represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms,

X represents one selected from the group consisting of a hydrogen atom, alkyl groups having 1 to 10 carbon atoms, alkenyl groups having 2 to 10 carbon atoms, and groups represented by the formula: —COOR⁴ (R⁴ has the same meaning as that of R², and may be the same as R² or different from R²), and

Y represents one selected from the group consisting of a hydrogen atom, alkyl groups having 1 to 10 carbon atoms, alkenyl groups having 2 to 10 carbon atoms, and groups represented by the formula: —COOR⁵ (R⁵ has the same meaning as that of R², and may be the same as R² or different from R²)]

in a carboxylic acid having 1 to 5 carbon atoms with a catalyst being used, to thereby obtain the carboxylic anhydride, wherein

the catalyst is a homogeneous acid catalyst having an acid dissociation constant (pKa) of −6.5 or lower and a boiling point of 100° C. or higher, the acid dissociation constant (pKa) being determined by a quantum chemistry calculation based on a density functional method.

(Homogeneous Acid Catalyst)

The catalyst used in the present invention is a homogeneous acid catalyst having an acid dissociation constant (pKa) of −6.5 or lower and a boiling point of 100° C. or higher, the acid dissociation constant (pKa) being determined by a quantum chemistry calculation based on a density functional method. As described above, one having such a sufficient acid strength that the acid dissociation constant (pKa) determined by a quantum chemistry calculation based on a density functional method is −6.5 or lower is used as the homogeneous acid catalyst. If the pKa exceeds the upper limit, the reaction rate is lowered, and the reaction takes a longer time, so that the heating of the product causes the formation of a colored component, and the color development in the product cannot be sufficiently suppressed. From the same viewpoint, the acid dissociation constant (pKa) of the homogeneous acid catalyst determined by a quantum chemistry calculation based on a density functional method is more preferably −7.0 or lower, and the acid dissociation constant (pKa) further preferably takes a value smaller than −8.0. Note that, in the present invention, a value calculated by a quantum chemistry calculation based on a density functional method (DFT method) is employed as the value of the “acid dissociation constant (pKa),” and such a value is employed as a criterion of the acid strength. Hereinafter, a method for calculating an “acid dissociation constant (pKa)” according to the present invention is described.

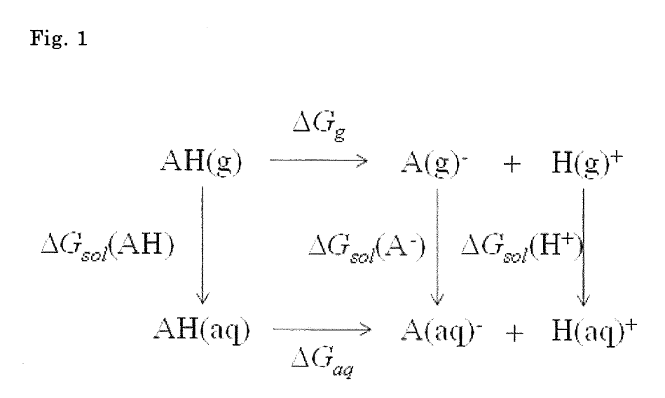

As the method for calculating an “acid dissociation constant (pKa)” according to the present invention, a method of calculation by a quantum chemistry calculation based on a density functional method (DFT method) is employed as described above. In the quantum chemistry calculation, structure optimization and frequency calculation of the acid catalyst are conducted by using software (trade name: Gaussian 09) manufactured by GAUSS IAN as quantum chemistry calculation software on a computer at the B3LYP/6-311++G(d,p) level. In addition, for the quantum chemistry calculation, thermodynamic quantities shown in a reaction scheme of an acid dissociation reaction (dissociation reaction of a proton) of an acid catalyst (an acid represented by the formula: AH) in FIG. 1 are calculated. Note that, in FIG. 1, AH represents an acid (acid catalyst), A⁻ represents an ion of the acid, and H⁺ represents a hydrogen ion (proton). In addition, in FIG. 1, the formula: AH(g)→A(g)⁻+H(g)⁺ represents the dissociation reaction of a proton in gas phase, and the formula: AH(aq)→A(aq)⁻+H(aq)⁺ represents a dissociation reaction of a proton in water. In addition, on the basis of the following calculation formula (1):

$$\begin{matrix}[ Math . 1 ] & \\ \begin{matrix}Δ {G}_{aq} = Δ {G}_{g} - Δ {G}_{aq} ( AH ) + \\ Δ {G}_{aq} ( {A}^{-} ) + Δ {G}_{aq} ( {H}^{+} ) \\ = Δ {G}_{g} + ΔΔ {G}_{aq} ,\end{matrix} & [ Calculation Formula ( 1 ) ]\end{matrix}$$

the change in free energy (ΔG_{g}) in the dissociation reaction of a proton in gas phase is first determined by using the above-described software, and then the change in free energy (ΔΔG_{aq}) in water is calculated by a PCM (polarizable continuum model) method to determine the change in free energy (ΔGaq) in the dissociation reaction of a proton in water. Subsequently, a calculation is made on the basis of the result (the value of ΔG_{aq}) by the following calculation formula (2):

$$\begin{matrix}[ Math . 2 ] & \\ pKa = \frac{Δ {G}_{aq}}{2.303 RT} & [ Calculation Formula ( 2 ) ]\end{matrix}$$

to calculate an acid dissociation constant (pKa) in water. Note that, for the calculation, ΔGₛₒₗ(AH) in FIG. 1 is the same as ΔG_{aq}(AH) in the calculation formula (1), ΔGₛₒₗ (A⁻) in FIG. 1 is the same as ΔG_{aq} (A⁻) in the calculation formula (1), and ΔGₛₒₗ(H⁺) in FIG. 1 is the same as ΔG_{aq}(H⁺) in the calculation formula (1). In addition, for the above-described calculation, the calculation is made supposing that the gas constant (R) is 1.9872 cal/mol·K, the temperature is 298.15 K, and further the pressure condition is a standard atmospheric pressure condition. In the present invention, the value of the acid dissociation constant (pKa) in water calculated as described above is employed as the value of the acid dissociation constant (pKa) determined by the quantum chemistry calculation based on the density functional method (DFT method).

In addition, in the present invention, one having a boiling point of 100° C. or higher is used as the homogeneous acid catalyst. If the boiling point is lower than 100° C., the catalyst has a lower boiling point than the lower carboxylic acid serving as the solvent, and hence is more likely to evaporate from the system during the reaction. Hence, a product in which color development is sufficiently suppressed cannot be produced efficiently. In addition, the boiling point of the homogeneous acid catalyst is more preferably 118 to 290° C., and further preferably 150 to 210° C. If the boiling point is lower than the lower limit, the boiling point is lower than that of the lower carboxylic acid serving as the solvent, and the catalyst is more likely to evaporate from the system during the reaction. Meanwhile, if the boiling point exceeds the upper limit, the mass of the catalyst used for the reaction tends to increase with the increase in molecular weight of the catalyst. Note that the “boiling point” herein refers to the boiling point (normal boiling point) at a pressure of 1 atm.

In addition, the molecular weight of the homogeneous acid catalyst is not particularly limited, and is preferably 1000 or less, and more preferably 600 or less. If the molecular weight exceeds the upper limit, the weight of the catalyst tends to increase when the catalyst is added in equivalents necessary for the reaction, so that such a molecular weight is disadvantageous in terms of costs.

In addition, the homogeneous acid catalyst is not particularly limited, as long as the homogeneous acid catalyst has an acid dissociation constant (pKa) of −6.5 or lower and has a boiling point of 100° C. or higher, the acid dissociation constant (pKa) being determined by the quantum chemistry calculation based on the density functional method. From known homogeneous acid catalysts, one satisfying the above-described conditions (the conditions that the pKa is −6.5 or lower, and the boiling point is 100° C. or higher) may be selected, as appropriate, and used. The homogeneous acid catalyst is preferably trifluoromethanesulfonic acid, tetrafluoroethanesulfonic acid, pentafluoroethanesulfonic acid, heptafluoropropanesulfonic acid, heptafluoroisopropanesulfonic acid, nonafluorobutanesulfonic acid, heptafluorodecanesulfonic acid, bis(nonafluorobutanesulfonyl)imide, N,N-bis(trifluoromethanesulfonyl)imide, or chlorodifluoroacetic acid, more preferably trifluoromethanesulfonic acid, tetrafluoroethanesulfonic acid, nonafluorobutanesulfonic acid, or chlorodifluoroacetic acid, and further preferably trifluoromethanesulfonic acid or tetrafluoroethanesulfonic acid from the viewpoints of the acid strength (the acid dissociation constant) and the availability. Note that one of these homogeneous acid catalysts may be used alone, or two or more thereof may be used in combination.

In addition, the amount of the homogeneous acid catalyst used is not particularly limited, and is preferably such an amount that the acid amount of moles of the homogeneous acid catalyst is 0.001 to 2.00 mole equivalents (more preferably 0.01 to 1.00 mole equivalents) to the amount (amount of moles) of the compound represented by the general formula (1) used. If the amount of the homogeneous acid catalyst used is less than the lower limit, the reaction rate tends to be lowered. Meanwhile, if the amount of the homogeneous acid catalyst used exceeds the upper limit, it is difficult to further improve the effect obtained by using the catalyst, and rather the economical efficiency tends to be lowered. Note that the acid amount of moles the homogeneous acid catalyst herein is the amount of moles in terms of the functional groups (for example, sulfonic acid groups (sulfo groups), carboxylic acid groups (carboxyl groups), or the like) in the homogeneous acid catalyst.

In addition, the amount of the homogeneous acid catalyst used is preferably 0.1 to 200 parts by mass, and more preferably 1 to 100 parts by mass relative to 100 parts by mass of the compound represented by the general formula (1). If the amount of the homogeneous acid catalyst used is less than the lower limit, the reaction rate tends to be lowered. Meanwhile, if the amount of the homogeneous acid catalyst used exceeds the upper limit, formation of side reaction products tends to occur more easily.

(Raw Material Compound)

The raw material compound used in the present invention is a compound (a carboxylic acid compound or a carboxylic acid ester compound) represented by the following general formula (1):

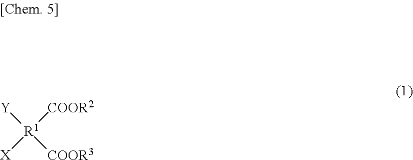

[in the formula (1),

R¹ is a tetravalent organic group having at least two carbon atoms adjacent to each other, and groups represented by the formulae: —COOR² and —COOR³ are bonded to one and the other of the two adjacent carbon atoms, respectively,

R² and R³, which may be the same or different, each represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms,

X represents one selected from the group consisting of a hydrogen atom, alkyl groups having 1 to 10 carbon atoms, alkenyl groups having 2 to 10 carbon atoms, and groups represented by the formula: —COOR⁴ (R⁴ has the same meaning as that of R², and may be the same as R² or different from R²), and

Y represents one selected from the group consisting of a hydrogen atom, alkyl groups having 1 to 10 carbon atoms, alkenyl groups having 2 to 10 carbon atoms, and groups represented by the formula: —COOR⁵ (R⁵ has the same meaning as that of R², and may be the same as R² or different from R²)].

R¹ in the general formula (1) is a tetravalent organic group having at least two carbon atoms adjacent to each other. In other words, R¹ is not particularly limited, as long as R¹ is a tetravalent organic group having at least two carbon atoms adjacent to each other and having four bonds through which the groups represented by the formulae: X, Y, COOR², and COOR³ are bonded. Examples of the tetravalent organic group include optionally heteroatom-containing tetravalent chainlike saturated hydrocarbon groups, optionally heteroatom-containing tetravalent cyclic saturated hydrocarbon groups, optionally heteroatom-containing tetravalent chainlike unsaturated hydrocarbon groups, optionally heteroatom-containing tetravalent cyclic unsaturated hydrocarbon groups, and the like. In addition, examples of organic groups which may be preferably used as R¹ include organic groups represented by the following general formulae (101) to (115):

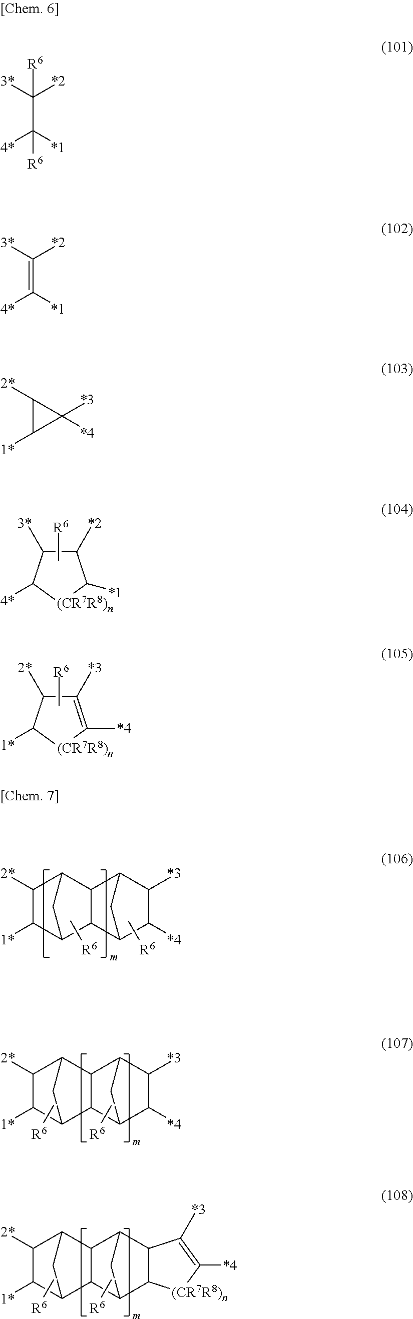

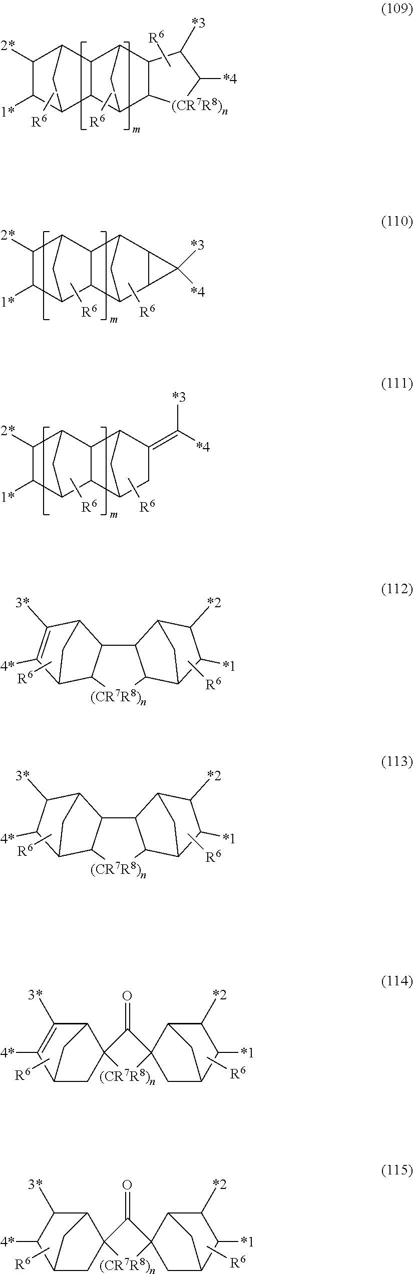

[in the formulae (101) to (115), *1 represents a bond to COOR² in the formula (1), *2 represents a bond to COOR³ in the formula (1), *3 represents a bond to X in the formula (1),*4 represents a bond to Y in the formula (1), R⁶s, R⁷, and R⁸ each independently represent one selected from the group consisting of a hydrogen atom, alkyl groups having 1 to 10 carbon atoms, and a fluorine atom, n represents an integer of 0 to 12, and m represents an integer of 0 to 5].

The number of carbon atoms of the alkyl group which may be selected as each of R⁶s in the general formulae (101) to (115) is 1 to 10. If the number of carbon atoms of the alkyl group exceeds the upper limit, the production and the purification tend to be difficult. In addition, the number of carbon atoms of the alkyl group which may be selected as each R⁶ is preferably 1 to 5, and more preferably 1 to 3 from the viewpoints of ease of production and purification. In addition, the alkyl group which may be selected as each R⁶ may be linear or branched. In addition, R⁶s in the general formulae (101) to (115) are each independently more preferably a hydrogen atom or an alkyl group having 1 to 10 carbon atoms from the viewpoints of ease of production and purification. Especially, R⁶s in the general formulae (101) to (115) are each independently more preferably a hydrogen atom, a methyl group, an ethyl group, a n-propyl group, or an isopropyl group, and particularly preferably a hydrogen atom or a methyl group from the viewpoints that the raw material is readily available and the purification is easier. In addition, multiple R⁶s in the formula are particularly preferably the same from the viewpoints of ease of production and purification and the like.

In addition, the alkyl group having 1 to 10 carbon atoms which may be selected as each of R⁷s and R⁸s in the general formulae (101) to (115) is the same as the alkyl group having 1 to 10 carbon atoms which may be selected as each R⁶. Among the above-described substituents, the substituent which may be selected as each of R⁷s and R⁸s is preferably a hydrogen atom or an alkyl group having 1 to 10 carbon atoms (more preferably 1 to 5, and further preferably 1 to 3 carbon atoms), and particularly preferably a hydrogen atom or a methyl group from the viewpoint of ease of the production and purification of the raw material compound.

In addition, in the general formulae (101) to (115), n represents an integer of 0 to 12. If the value of n exceeds the upper limit, the raw material compounds represented by the general formulae (101) to (115) are difficult to purify. In addition, in the general formulae (101) to (115), the upper limit value of the numeric value range of n is more preferably 5, and particularly preferably 3 from the viewpoint that the raw material compounds are easier to purify. In addition, in the general formulae (101) to (115), the lower limit value of the numeric value range of n is more preferably 1, and particularly preferably 2, from the viewpoint of the stability of the raw materials. As described above, n in the general formulae (101) to (115) is particularly preferably an integer of 2 or 3.

Moreover, m in each of the general formulae (106) to (111) represents an integer of 0 to 5. If the value of m exceeds the upper limit, the compounds represented by the general formulae (106) to (111) are difficult to produce and purify. In addition, the upper limit value of the numeric value range of m in the general formulae (106) to (111) is more preferably 3, and particularly preferably 1 from the viewpoint of ease of production and purification. In addition, the lower limit value of the numeric value range of m in the general formulae (106) to (111) is particularly preferably 0 from the viewpoint of ease of production and purification. As described above, m in the general formulae (106) to (111) is particularly preferably an integer of 0 or 1.

In addition, in the compound represented by the general formula (1), R² and R³, which may be the same or different, each represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 2 0 carbon atoms, and aralkyl groups having 7 to 20 carbon atoms.

The alkyl group which may be selected as each of R² and R³ in the general formula (1) is an alkyl group having 1 to 10 carbon atoms. If the number of carbon atoms of the alkyl group exceeds 10, the purification is difficult. In addition, the number of carbon atoms of the alkyl group which may be selected as each of R² and R³ is more preferably 1 to 5, and further preferably 1 to 3 from the viewpoint that the purification is easier. In addition, the alkyl group which may be selected as each of R² and R³ may be linear or branched.

In addition, the cycloalkyl group which may be selected as each of R² and R³ in the general formula (1) is a cycloalkyl group having 3 to 10 carbon atoms. If the number of carbon atoms of the cycloalkyl group exceeds 10, the purification is difficult. In addition, the number of carbon atoms of the cycloalkyl group which may be selected as each of R² and R³ is more preferably 3 to 8, and further preferably 5 to 6 from the viewpoint that the purification is easier.

Moreover, the alkenyl group which may be selected as each of R² and R³ in the general formula (1) is an alkenyl group having 2 to 10 carbon atoms. If the number of carbon atoms of the alkenyl group exceeds 10, the purification is difficult. In addition, the number of carbon atoms of the alkenyl group which may be selected as each of R² and R³ is more preferably 2 to 5, and further preferably 2 or 3 from the viewpoint that the purification is easier.

In addition, the aryl group which may be selected as each of R² and R³ in the general formula (1) is an aryl group having 6 to 20 carbon atoms. If the number of carbon atoms of the aryl group exceeds 20, the purification is difficult. In addition, the number of carbon atoms of the aryl group which may be selected as each of R² and R³ is more preferably 6 to 10, and further preferably 6 to 8 from the viewpoint that the purification is easier.

In addition, the aralkyl group which may be selected as each of R² and R³ in the general formula (1) is an aralkyl group having 7 to 20 carbon atoms. If the number of carbon atoms of the aralkyl group exceeds 20, the purification is difficult. In addition, the number of carbon atoms of the aralkyl group which may be selected as each of R² and R³ is more preferably 7 to 10, and further preferably 7 to 9 from the viewpoint that the purification is easier.

Moreover, from the viewpoint that the purification is easier, R² and R³ in the general formula (1) is each independently preferably a hydrogen atom, a methyl group, an ethyl group, a n-propyl group, an isopropyl group, a n-butyl group, an isobutyl group, a sec-butyl group, a t-butyl group, a 2-ethylhexyl group, a cyclohexyl group, an allyl group, a phenyl group, or a benzyl group, and particularly preferably a methyl group. Note that R² and R³ in the general formula (1) may be the same or different, and are more preferably the same from the viewpoint of the synthesis.

In addition, in the compound represented by the general formula (1), the groups represented by the formulae: —COOR² and —COOR³ have to be bonded to one and the other of the adjacent two carbon atoms, respectively, in the tetravalent organic group. Now, a description is given by taking a case where R¹ is an organic group represented by any of the general formulae (101) to (115) as an example. In the raw material compound, the group represented by the formulae: COOR² is bonded to one of the adjacent carbons in the organic group and the group represented by the formulae: COOR³ is bonded to the other one of the adjacent carbons through the bonds (for example, *1 and *2), respectively. As described above, it is necessary to use, as the raw material compound, one in which the groups represented by the formulae: —COOR² and —COOR³ are introduced to one and the other of the adjacent two carbon atoms, respectively. This makes it possible to form the acid anhydride.

In addition, in the general formula (1), X represents one selected from the group consisting of a hydrogen atom, alkyl groups having 1 to 10 carbon atoms, alkenyl groups having 2 to 10 carbon atoms, and groups represented by the formula: —COOR⁴ (R⁴ has the same meaning as that of R², and may be the same as R² or different from R²).

If the number of carbon atoms of the alkyl group which may be selected as X in the general formula (1) exceeds the upper limit, the production and the purification tend to be difficult. In addition, the number of carbon atoms of the alkyl group which may be selected as X is preferably 1 to 6, and more preferably 1 to 4 from the viewpoints of ease of production and purification. In addition, the alkyl group which may be selected as X may be linear or branched.

In addition, if the number of carbon atoms of the alkenyl group which may be selected as X in the general formula (1) exceeds the upper limit, the production and the purification tend to be difficult. In addition, the number of carbon atoms of the alkenyl group which may be selected as X is preferably 2 to 6, and more preferably to 4 from the viewpoints of ease of production and purification. In addition, the alkenyl group which may be selected as X may be linear or branched.

In addition, in the group represented by the formula: —COOR⁴ which may be selected as X in the general formula (1), R⁴ is the same as R² (one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms), and preferred ones thereof are also the same as those of R².

X is more preferably a group represented by the formula: —COOMe or —COOEt.

In addition, in the general formula (1), Y represents one selected from the group consisting of a hydrogen atom, alkyl groups having 1 to 10 carbon atoms, alkenyl groups having 2 to 10 carbon atoms, and groups represented by the formula: —COOR⁵ (R⁵ has the same meaning as that of R², and may be the same as R² or different from R²). The alkyl group having 1 to 10 carbon atoms and the alkenyl group having 2 to 10 carbon atoms each of which may be selected as Y in the formula (1) are the same as those described for X. In addition, R⁵ in the group represented by the formula: —COOR⁵ which may be selected as Y in the general formula (1) is the same as R² (one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 2 0 carbon atoms, and aralkyl groups having 7 to 2 0 carbon atoms), and preferred ones thereof are also the same as those of R². Y is more preferably a group represented by the formula: —COOMe or —COOEt.

In addition, when the raw material compound represented by the general formula (1) contains a group (s) represented by the formulae: —COOR⁴ and/or —COOR⁵, R², R³, R⁴, and R⁵ may be the same or different, and are more preferably the same from the viewpoint of the synthesis of the raw material compound.

In addition, in the raw material compound represented by the general formula (1), X and Y are preferably a group represented by the formula: —COOR⁴ and a group represented by —COOR⁵, respectively, from the viewpoint of ease of production and purification. As described above, the raw material compound represented by the general formula (1) is preferably a tetracarboxylic acid compound or a tetracarboxylic acid ester compound.

In addition, examples of the raw material compound represented by the general formula (1) include compounds (examples of compounds of the formula (1), in which X and Y are both hydrogen atoms) represented by the following general formulae (1-1) to (1-16):

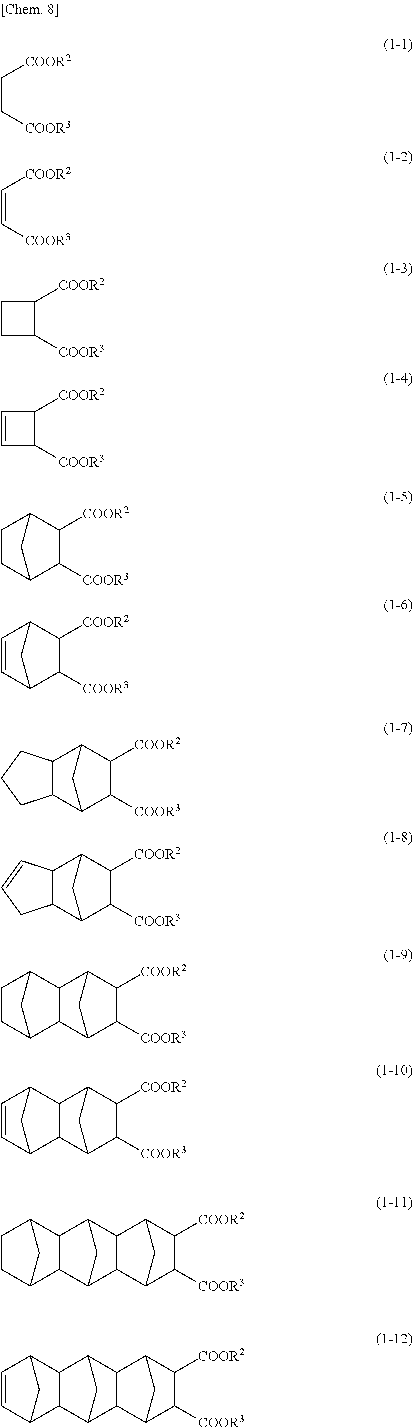

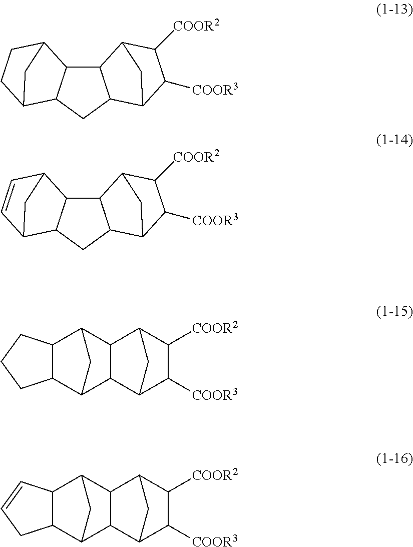

[in the formulae, R² and R³ have the same meanings as those of R² and R³ described for the general formula (1)];

compounds (examples of compounds of the formula (1), in which one of X and Y is a hydrogen atom, and the other is an alkyl group or an alkenyl group) represented by the following general formulae (1-17) to (1-19):

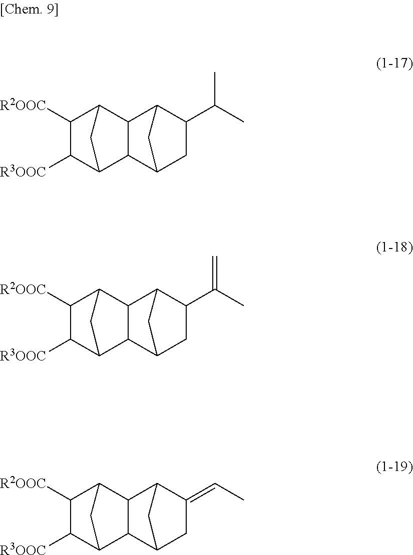

[in the formula, R² and R³ have the same meanings as those of R² and R³ described for the general formula (1)];

compounds (examples of compounds of the formula (1), in which X is a group represented by the formula: —COOR⁴, and Y is a group represented by the formula: —COOR⁵) represented by the following general formulae (1-20) to (1-26):

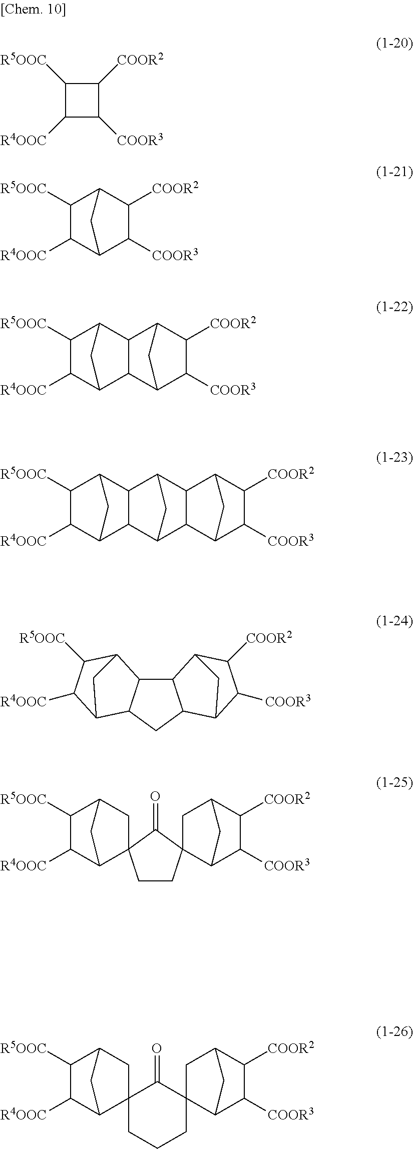

[in the formulae, R², R³, R⁴, and R⁵ have the same meanings as those of R², R³, R⁴, R⁵ described for the general formula (1)]; and the like.

In addition, from the viewpoint that a carboxylic anhydride can be produced which is preferably usable as a material (monomer) for forming a polyimide having an excellent heat resistance and a sufficiently low linear expansion coefficient, the raw material compound represented by the general formula (1) is preferably a spiro compound represented by the following general formula (2):

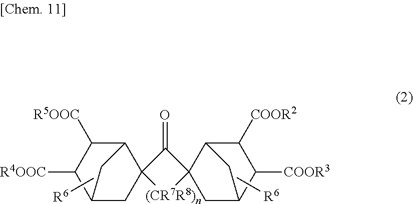

[in the formula (2), R², R³, R⁴, and R⁵ have the same meanings as those of R², R³, R⁴, and R⁵ described for the general formula (1) (and preferred ones thereof are also the same), and R⁶s, R⁷, and R⁸, which may be the same or different, each represent one selected from the group consisting of a hydrogen atom, alkyl groups having 1 to 10 carbon atoms, and a fluorine atom, and n represents an integer of 0 to 12].

Note that R⁶s, R⁷, and R⁸ in the general formula (2) are the same as R⁶s, R⁷, and R⁸ in the general formulae (101) to (115), and preferred ones thereof are also the same.

In addition, a method for preparing the raw material compound is not particularly limited, and a known method can be used, as appropriate. For example, when the compound (spiro compound) represented by the general formula (2) is used as the raw material compound, the method for preparing a spiro compound disclosed in International Publication No. WO 2011/099518 may be used, as appropriate.

(Lower Carboxylic Acid)

In the present invention, a carboxylic acid having 1 to 5 carbon atoms (hereinafter, sometimes simply referred to as “lower carboxylic acid”) is used. If the number of carbon atoms of the lower carboxylic acid exceeds the upper limit, the production and purification are difficult. In addition, examples of the lower carboxylic acid include formic acid, acetic acid, propionic acid, butyric acid, and the like, of which formic acid, acetic acid, and propionic acid are preferable, and formic acid and acetic acid are more preferable from the viewpoint of ease of production and purification. One of these lower carboxylic acids may be used alone, or two or more thereof may be used in combination.

In addition, the amount of the lower carboxylic acid (for example, formic acid, acetic acid, or propionic acid) used is not particularly limited, and is preferably such that the amount of moles of the lower carboxylic acid is 4 to 100 times that of the raw material compound represented by the general formula (1). If the amount of the lower carboxylic acid (formic acid, acetic acid, propionic acid, or the like) used is less than the lower limit, the reaction rate tends to be lowered. Meanwhile, if the amount of the lower carboxylic acid exceeds the upper limit, the yield tends to decrease. In addition, the amount of the raw material compound represented by the general formula (1) contained in the lower carboxylic acid is preferably 1 to 40% by mass, and more preferably 2 to 30% by mass.

(Heating Step)

In the present invention, a step (heating step) is performed in which the raw material compound is heated in the lower carboxylic acid with a catalyst being used. Note that, the homogeneous acid catalyst is used as the catalyst in the present invention. Accordingly, the heating step is a step of heating the raw material compound in the lower carboxylic acid with the homogeneous acid catalyst being used.

In this heating step, another solvent may be used by being added to the lower carboxylic acid. Examples of the solvent (another solvent) include aromatic solvents such as benzene, toluene, xylene, and chlorobenzene; ether-based solvents such as ether, THF, and dioxane; ester-based solvents such as ethyl acetate; hydrocarbon-based solvents such as hexane, cyclohexane, heptane, and pentane; nitrile-based solvents such as acetonitrile and benzonitrile; halogen-containing solvents such as methylene chloride and chloroform; ketone-based solvents such as acetone and MEK; and amide-based solvents such as DMF, NMP, DMI, and DMAc.

Meanwhile, in this heating step, acetic anhydride may be used with the lower carboxylic acid. The use of acetic anhydride as described above makes it possible to react the acetic anhydride with water produced during the reaction to form acetic acid, so that the water produced during the reaction can be removed efficiently. In addition, when acetic anhydride is used as described above, the amount of the acetic anhydride used is not particularly limited, and is preferably such that the amount of moles of the acetic anhydride used is 4 to 100 times that of the raw material compound represented by the general formula (1). If the amount of the acetic anhydride used is less than the lower limit, the reaction rate tends to be lowered. Meanwhile, if the amount of the acetic anhydride used exceeds the upper limit, the yield tends to decrease.

In addition, a temperature condition under which the raw material compound is heated in the lower carboxylic acid is not particularly limited. The upper limit of the heating temperature is preferably 180° C. (more preferably 150° C., further preferably 140° C., and particularly preferably 130° C.), while the lower limit of the heating temperature is preferably 80° C. (more preferably 100° C., and further preferably 110° C.). The temperature range (temperature condition) for the heating is preferably 80 to 180° C., more preferably 80 to 150° C., further preferably 100 to 140° C., and particularly preferably 110 to 130° C. If the temperature condition is lower than the lower limit, the reaction tends not to proceed sufficiently, so that the target carboxylic anhydride cannot be produced sufficiently efficiently. Meanwhile, if the temperature condition exceeds the upper limit, the catalytic activity tends to be lowered. In addition, the heating temperature is preferably set to a temperature lower than the boiling point of the homogeneous acid catalyst within the range of the above-described temperature condition. By setting the heating temperature as described above, the product can be obtained more efficiently.

In addition, a pressure condition (the pressure condition during the reaction) under which the raw material compound is heated in the lower carboxylic acid is not particularly limited, and the heating may be conducted under normal pressure, under a pressurized condition, or under a reduced pressure condition. Under any one of these conditions, the reaction can be caused to proceed. For this reason, when, for example, reflux is employed in the heating step without controlling the pressure particularly, the reaction may be carried out under a pressurized condition of the vapor of the lower carboxylic acid serving as the solvent or the like. In addition, the pressure condition is preferably 0.001 to 10 MPa, and further preferably 0.1 to 1.0 MPa. If the pressure condition is lower than the lower limit, the lower carboxylic acid tends to be gasified. Meanwhile, if the pressure condition exceeds the upper limit, the lower carboxylic acid ester formed by the reaction tends not to evaporate, so that the equilibrium reaction of the esterification tends to be difficult to proceed. In addition, an atmospheric gas in which the raw material compound is heated in the lower carboxylic acid is not particularly limited, and, for example, may be air or an inert gas (nitrogen, argon, or the like). Note that, to efficiently evaporate the lower carboxylic acid ester and water formed by the reaction and to cause the reaction to proceed more efficiently (to shift the equilibrium react ion of the esterification to the product side), the gas (desirably, an inert gas such as nitrogen or argon) may be bubbled, or stirring may be conducted, while the gas is being passed through the gas phase portion of a reactor (reaction vessel).

In addition, a heating time for which the raw material compound is heated in the lower carboxylic acid is not particularly limited, and is preferably 0.5 to 100 hours, and more preferably 1 to 50 hours. If the heating time is less than the lower limit, the reaction tends to proceed so insufficiently that a sufficient amount of the carboxylic anhydride cannot be produced. Meanwhile, if the heating time exceeds the upper limit, the reaction tends not to proceed any further, so that the production efficiency is lowered, and the economical efficiency and the like are lowered.

In addition, when the raw material compound is heated in the lower carboxylic acid, the reaction may be caused to proceed while the lower carboxylic acid to which the raw material compound is introduced is being stirred, from the viewpoint that the reaction is caused to proceed uniformly.

In addition, in the step (heating step) of heating the raw material compound in the lower carboxylic acid with the homogeneous acid catalyst being used, an acid anhydride group represented by the following general formula (3):

*⁵—CO—O—OC—*⁶ (3)

[in the formula (3), *5 and *6 represents bonds to the carbon atoms to which the groups represented by the formulae: —COOR² and —COOR³ (when X and Y are groups represented by —COOR⁴ and —COOR⁵, the groups represented by —COOR² and —COOR³ and the groups represented by —COOR⁴ and —COOR⁵ depending on the case) in the raw material compound are bonded, respectively],

is formed from at least the groups represented by the formulae: —COOR² and —COOR³ in the raw material compound (also from the groups represented by —COOR⁴ and —COOR⁵ depending on the case, when X and Y are the groups represented by —COOR⁴ and —COOR⁵, respectively,), so that a carboxylic anhydride is formed. Now, the reaction by which the carboxylic anhydride is formed is briefly described by taking a case where a spiro compound represented by the general formula (2) is used as an example. This reaction is as represented by the following reaction formula (I):

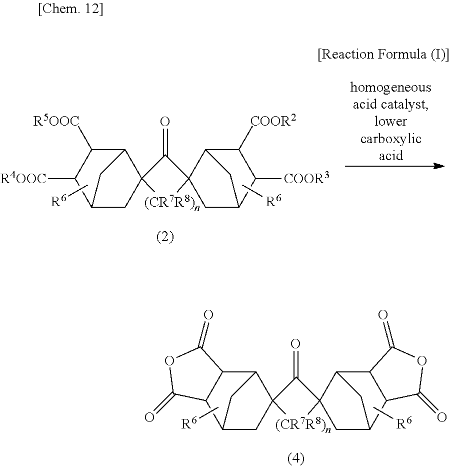

[in the reaction formula (I), R², R³, R⁴, R⁵, R⁶s, R⁷, and R⁸ have the same meanings as those of R², R³, R⁴, R⁵, R⁶s, R⁷, and R⁸ described for the general formula (2) (and preferred ones thereof are also the same)]. When the spiro compound represented by the general formula (2) is used as the raw material compound as described above, the tetracarboxylic anhydride as represented by the general formula (4) is obtained. In addition, similarly, examples of reactions are shown in which a compound represented by the general formula (1-5), a compound represented by the general formula (1-21), and a compound represented by the general formula (1-22) are used as the raw material compounds. The reactions are as represented by the following reaction formulae (II), (III), and (IV):

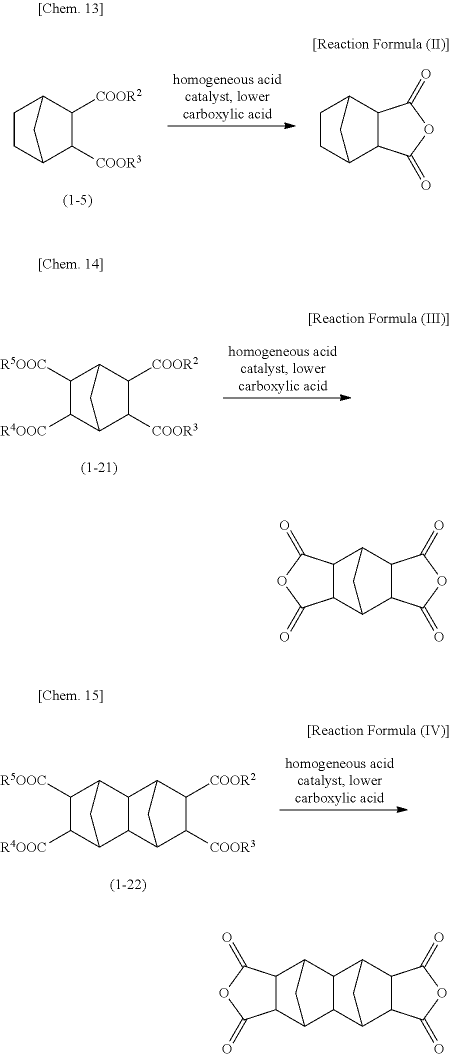

[in the reaction formulae (II) to (IV), R², R³, R⁴, and R⁵ have the same meanings as those of R², R³, R⁴, R⁵ described for the general formula (1) (and preferred ones thereof are also the same)].

As shown in these reaction formulae (I) to (IV), the acid anhydride group represented by the general formula (3) is formed in the above-described heating step from the ester groups and/or carboxylic acid groups (the groups represented by the formulae: —COOR² and —COOR³ (the group represented by —COOR⁴ and —COOR⁵ depending on the case)) bonded to the two adjacent carbon atoms in the raw material compound, so that the carboxylic anhydride is formed. Note that, in the heating step, the carboxylic anhydride, which is the final product, can be obtained as a precipitate (deposit or the like).

In addition, although it is not exactly clear, the reactions occurring in this heating step are presumably as follows. Specifically, a description is made while taking, as an example, a case (a preferred embodiment) where a compound represented by the general formula (1), in which X is a group represented by the formula: —COOR⁴, and Y is a group represented by the formula: —COOR⁵, X and Y are respectively connected to adjacent carbon atoms in the compound, and R², R³, R⁴, and R⁵ are each a group other than a hydrogen atom, is used as the raw material compound, and acetic acid is used as the lower carboxylic acid. In this case, the reaction is presumably as represented by the following reaction formulae (V) and (VI):

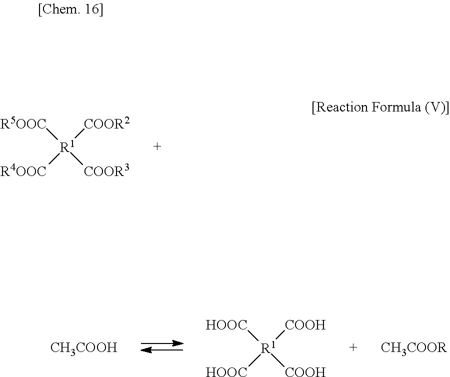

[in the reaction formula (V), R¹ has the same meaning as that of R¹ in the general formula (1), R², R³, R⁴, and R⁵ are the same as R², R³, R⁴, and R⁵ described for the general formula (1), except that they are other than hydrogen atoms, and R is any one of the groups R², R³, R⁴, and R⁵ in the raw material compound], and

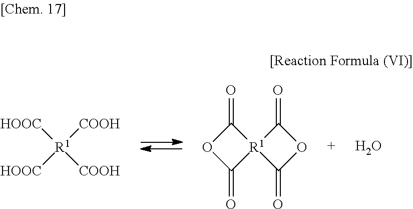

[in the reaction formula (VI), R¹ is the same as R¹ described for the general formula (1)]. Note that the reaction formula (V) represents a reaction in which the ester groups in the raw material compound are degraded to the carboxylic acid, and the reaction formula (VI) represents the subsequent reaction of the formation of the acid anhydride. In addition, presumably, the reaction represented by the reaction formula (V) in which the ester groups are degraded to the carboxylic acid, and the subsequent reaction represented by the reaction formula (VI) in which the acid anhydride is formed occur continuously. Note that when each of R², R³, R⁴, and R⁵ in the raw material compound is a hydrogen atom, the above-described reaction represented by the reaction formula (VI) proceeds in the heating step.

In addition, the reactions for forming the carboxylic anhydride exemplified by the reaction formulae (V) and (VI) are both equilibrium reactions. Note that the carboxylic anhydride formed by the reactions has an extremely low solubility in the lower carboxylic acid, and hence tends to be easily precipitated during the reaction. As described above, the carboxylic anhydride tends to be easily precipitated as a precipitate (deposit or the like) in the lower carboxylic acid during the above-described reaction. Hence, the above-described reaction in the solution tends to be in favor of the formation of the acid anhydride, so that the reaction proceeds more efficiently.

In addition, each of the reactions for forming the carboxylic anhydride as exemplified in the reaction formulae (V) and (VI) is an equilibrium reaction. Hence, when the raw material compound is an ester compound (for example, a raw material compound of the general formula (1), in which at least R² and/or R³ are other than hydrogen atoms, or the like), and the raw material compound is heated in the lower carboxylic acid, for example, the reaction in which the ester groups in the raw material compound are degraded to the carboxylic acid (a reaction as represented by the above-described reaction formula (V)) is preferably caused to proceed, while the formed lower carboxylic acid ester (an acetic acid ester represented by the formulae: CH₃COOR in the reaction as represented by the above-described reaction formula (V)) is being removed by distillation to the outside of the reaction system, and water produced during the reaction is preferably removed by distillation to the outside of the reaction system or removed by a reaction with another substance (for example, an acid anhydride of a lower carboxylic acid such as acetic anhydride) in the subsequent reaction of formation of the acid anhydride (a reaction as represented by the reaction (VI)), from the viewpoint of efficiently producing the target carboxylic anhydride.

From the viewpoint of performing the reaction of the degradation of the ester groups in the raw material compound to the carboxylic acid and the subsequent reaction of the formation of the acid anhydride more efficiently in the reactions in which the raw material compound is heated in the lower carboxylic acid to obtain the carboxylic anhydride as described above, a method may be employed in which the heating step comprises, for example: a step (I) of preparing a mixture liquid of the compound represented by the general formula (1), the lower carboxylic acid, and the homogeneous acid catalyst and heating the mixture liquid under reflux; and a step (II) of heating the mixture liquid, while removing vapor by distillation from the solution subjected to the reflux, and continuously adding the lower carboxylic acid in an amount which is equal to the reduced amount, to thereby obtain the carboxylic anhydride. This method makes it possible to remove the lower carboxylic acid ester and water produced in the step (II) as vapor to the outside of the system. Note that the degree of the progress of the reaction can be determined by checking the amount of the lower carboxylic acid ester compound (an acetic acid ester represented by the formulae: CH₃COOR in a reaction as represented by the above-described reaction formula (V)) contained in the vapor removed by distillation.

When the mixture liquid is produced in the step (I), the amount of moles of the lower carboxylic acid used is preferably 2 to 500 times (more preferably about 50 times) that of the compound represented by the general formula (1).

In addition, suppose a case where a raw material compound represented by the general formula (1), in which R² and/or R³ are other than hydrogen atoms, is used (or, in a case where X and Y are respectively the groups represented by the formulae: —COOR⁴ and —COOR⁵, suppose a case where a raw material compound in which R² and/or R³ and/or R⁴ and/or R⁵ are groups other than hydrogen atoms). In such a case, the continuous addition of the lower carboxylic acid to the solution subjected to the reflux, while removing the vapor by distillation in the step (II), makes it possible to completely convert the ester groups to which the groups other than hydrogen atoms are bonded to carboxylic acid groups (—COOH) (to convert R² and/or R³ and/or R⁴ and/or R⁵ which are groups other than hydrogen atoms to hydrogen atoms: the reaction is the conversion (substitution) of OR to (with) OH: carboxylic acid formation)). Dehydration condensation can be achieved by heating the thus obtained carboxylic acid compound, as it is, so that the carboxylic anhydride group can be formed in the continuous steps, and water produced during the formation of the carboxylic anhydride group can also be easily removed as vapor to the outside of the system. Hence, it is also possible to more efficiently produce the carboxylic anhydride. In addition, a method for removing the vapor by distillation in the step (II) is not particularly limited, and a known method can be used, as appropriate. For example, a method using a Liebig condenser or the like may be employed. Note that when vapor is removed by distillation as described above, it is preferable to remove the distillate components, after the carboxylic acid having 1 to 5 carbon atoms is separated from the vapor. A step of separating the carboxylic acid having 1 to 5 carbon atoms as described above can be easily archived by using, for example, a rectification tower. The separation of the carboxylic acid having 1 to 5 carbon atoms from the vapor as described above makes it possible to easily remove unnecessary water and the like as the vapor to the outside of the system, while enabling the carboxylic acid having 1 to 5 carbon atoms to be reused (for example, the carboxylic acid having 1 to 5 carbon atoms after the separation to be returned to the reaction system and reused). Hence, it is also possible to conduct the reaction further industrially efficiently.

In addition, from the viewpoint of more efficiently removing the lower carboxylic acid ester and water by distillation (remove) when the lower carboxylic acid ester compound and water formed in the step (II) are removed as the vapor by distillation to the outside of the system, it is preferable to add, to the lower carboxylic acid, a compound which undergoes an azeotropic phenomenon with the lower carboxylic acid ester compound and water. The azeotropy agent is not particularly limited, as long as the azeotropy agent does not react with any of the raw material compound, the lower carboxylic acid, and the homogeneous acid catalyst, and a known azeotropy agent can be used, as appropriate. As the azeotropy agent, it is preferable to use, for example, a hydrocarbon such as benzene, toluene, pentane, hexane, cyclohexane, heptane, or octane; an ether such as diethyl ether, propyl ether, or tetrahydrofuran; or a halogenated hydrocarbon such as methylene chloride, chloroform, or trichloroethane.

In addition, the temperature condition of the heating in the steps (I) and (II) is preferably 60° C. to 180° C., and more preferably 100° C. to 140° C. If the temperature of the heating under reflux is lower than the lower limit, the yield tends to decrease. Meanwhile, if the temperature of the heating under reflux exceeds the upper limit, by-products tend to increase and the color development tends to occur to lower the transparency. In addition, the heating time is preferably about 30 minutes to 24 hours.

In addition, from the viewpoint of efficiently conducting the reaction of the degradation of the ester groups in the raw material compound to the carboxylic acid and the subsequent reaction of the formation of the anhydride, a method may be employed in which the following steps (A) to (C) are carried out in the heating step in the reaction in which the raw material compound is heated in the lower carboxylic acid to obtain the carboxylic anhydride. Specifically, as the heating step, a heating step may be performed which comprises: a step (A) of preparing a mixture liquid of the compound represented by the general formula (1), the lower carboxylic acid, and the homogeneous acid catalyst, and heating the mixture liquid under reflux;

a step (B) of removing a portion of liquid in the mixture liquid by distillation under reduced pressure to concentrate the mixture liquid, adding again the lower carboxylic acid to the obtained liquid concentrate followed by heating under reflux, and then removing a portion of liquid in the obtained mixture liquid by distillation under reduced pressure for concentrating again to thereby obtain a liquid concentrate; and

a step (C) of adding the acetic anhydride together with the lower carboxylic acid (formic acid, acetic acid, propionic acid, or the like) to the liquid concentrate followed by heating under reflux, to thereby obtain a carboxylic anhydride.

By employing the heating step comprising the steps (A) to (C), the carboxylic anhydride can be obtained more efficiently from the raw material compound represented by the general formula (1). Now, a description is made while taking the reaction formulae (V) and (VI) as examples. The reaction (the reaction of the degradation of the ester groups in the raw material compound to the carboxylic acid) as represented by the reaction formula (V) proceeds in the steps (A) and (B), while the reaction (anhydride formation reaction) as represented by the reaction formula (VI) proceeds in the step (C).

In addition, when the method comprising these steps (A) to (C) is employed, it is preferable to carry out the step of adding the lower carboxylic acid to the liquid concentrate followed by concentration repeatedly (preferably carried out repeatedly once to five times) in the step (B), or it is preferable to employ a step of removing the formed lower carboxylic acid ester compound and water together with the lower carboxylic acid by distillation, and then continuously adding the lower carboxylic acid in the reduced amount as the step (B). When a raw material compound of the general formula (1), in which R² and/or R³ are groups other than hydrogen atoms, is used (when a raw material compound in which R² and/or R³ and/or R⁴ and/or R⁵ are groups other than hydrogen atoms is used in a case where X and Y are groups represented by the formulae: —COOR⁴ and —COOR⁵, respectively), this step (B) makes it possible to completely convert the ester groups to which the groups other than hydrogen atoms are bonded to carboxylic acid groups (—COOH) (to convert R² and/or R³ and/or R⁴ and/or R⁵ which are the groups other than hydrogen atoms to hydrogen atoms: the reaction is conversion (substitution) of OR to (with) OH) can be carried out more efficiently, and the carboxylic anhydride can be obtained more efficiently by the step (C) carried out after the step (B). Note that the degree of the progress of the reaction in the step (B) can be determined by checking the amount of the lower carboxylic acid ester compound (an acetic acid ester represented by the formulae: CH₃COOR in a reaction as represented by the above-described reaction formula (V)) contained in the vapor removed by distillation.

Moreover, when the mixture liquid is produced in the step (A), the amount of moles of the lower carboxylic acid used is preferably 2 to 500 times (more preferably about 50 times) that of the compound represented by the general formula (1). In addition, the amount of the lower carboxylic acid (formic acid or the like) added to the liquid concentrate in the steps (B) and (C) is preferably about the same as the amount of the liquid removed by distillation during the concentration.

In addition, a method for concentrating the mixture liquid in the step (B) (for the removal by distillation under reduced pressure) is not particularly limited, and a known method can be employed, as appropriate. In addition, the temperature condition for the heating under reflux in the steps (A) to (C) is preferably 60° C. to 180° C., and more preferably 100° C. to 140° C. If the temperature of the heating under reflux is lower than the lower limit, the yield tends to decrease. Meanwhile, if the temperature of the heating under reflux exceeds the upper limit, by-products tend to increase and color development tends to more likely to occur. In addition, the time for the heating under reflux is preferably about 30 minutes to 24 hours.

Although the homogeneous acid catalyst is used in the heating step in the present invention as described above, a carboxylic anhydride can be efficiently obtained in which color development is sufficiently suppressed. Note that, since the homogeneous acid catalyst is used in the present invention, the present invention basically does not require a pretreatment for separating the crystals from the catalyst for collecting the crystals in contrast to a case where a heterogeneous catalyst is used, and makes it possible to easily collect the crystals by only a simple step such as filtration. Hence, the carboxylic anhydride can be produced more efficiently. In addition, since the homogeneous acid catalyst is used in the present invention, the decrease in amount (loss) of the crystals can also be sufficiently prevented during the step of separating the crystals from the catalyst in contrast to a case where a heterogeneous catalyst is used, and hence it is also possible to produce the target compound in a sufficient percentage yield.

Note that after a crude product of the carboxylic anhydride is obtained from the raw material compound represented by the general formula (1) as described above, the crude product may be subjected to a purification step such as recrystallization or sublimation, as appropriate. Such a purification step makes it possible to obtain the carboxylic anhydride with a higher purity. A method for the purification is not particularly limited, and a known method can be employed, as appropriate.

### EXAMPLES

Hereinafter, the present invention is described more specifically on the basis of Examples and Comparative Examples; however, the present invention is not limited to Examples below.

### Example 1

First, to a flask having a capacity of 300 mL and equipped with a reflux tube, a solution was added in which 10 g of a norbornane tetracarboxylic acid tetramethyl ester (norbornane-2-spiro-α-cyclopentanone-α′-spiro-2″-norbornane-5,5″,6,6″-tetracarboxylic acid tetramethyl ester; molecular weight: 476.52; raw material compound) represented by the following general formula (5) was dissolved in 190 g of acetic acid:

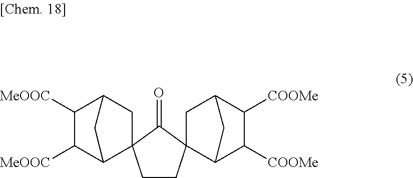

After that, 0.38 g of tetrafluoroethanesulfonic acid (HCF₂CF₂SO₃H, boiling point: 210° C.) was added as a homogeneous acid catalyst to the solution. Note that the raw material compound was prepared by employing the same method as the method described in Example 1 of International Publication No. WO2011/099518. In addition, the amount of the acid catalyst used was such that the mole ratio of the raw material compound to the acid catalyst ([amount of moles of raw material compound]:[amount of moles of functional groups (sulfonic acid) in the catalyst]) was 1:0.1 (the amount of moles of the acid serving as the catalyst was 0.1 mole equivalents to the raw material compound), and also such that the mass ratio of the acid catalyst was 3.8 parts by mass relative to 100 parts by mass of the raw material compound. Moreover, to determine the acid strength of the tetrafluoroethanesulfonic acid, the “acid dissociation constant (pKa)” was calculated by employing the above-described method for calculating an “acid dissociation constant (pKa)” according to the present invention (calculated by the density functional method using the software (trade name: Gaussian 09) manufactured by GAUSSIAN). The acid dissociation constant (pKa) calculated was −9.5.

Next, the atmospheric gas in the flask was replaced with nitrogen, and then the solution was heated under a nitrogen stream under a condition of atmospheric pressure, while being stirred by using a magnetic stirrer. The solution was refluxed for 0.5 hours with the temperature inside the flask being kept at 118° C. by the heating (reflux step). After the reflux step, a step (hereinafter, referred to as “step (i)”) of removing the generated vapor by distillation using a Liebig condenser under a heated condition of 118° C., and simultaneously adding acetic acid to the flask by using a dropping funnel to keep the amount of liquid in the flask constant was performed. Note that, in this step (i), after 2 hours from the start of the removal of the vapor by distillation, the formation of a white deposit was observed in the liquid (in the reaction solution) in the flask. In addition, in the step (i), the distillate removed by distillation to the outside of the system was measured for the mass and analyzed with a gas chromatograph every hour to determine the degree of the progress of the reaction. Note that this analysis showed the presence of acetic acid, methyl acetate, and water in the distillate. In addition, the speed of the removal of the distillate in the step was measured, and the speed (rate) of the removal of the distillate was approximately 35 mL per hour. In addition, after 4 hours from the start of the removal of the vapor by distillation in step (i), the methyl acetate ceased to be distilled off. Hence, the heating was stopped, and the step (i) was completed. Note that the amount (total amount) of methyl acetate distilled off by the time 4 hours had passed since the removal by distillation was started was 5.5 g. In addition, the amount of acetic acid removed by distillation by the time methyl acetate ceased to be distilled off (by the time the reaction was completed) was 85 g.

After the step (i) was performed as described above, acetic acid was removed by distillation from the solution in the flask to obtain a liquid concentrate. Then, the liquid concentrate was subjected to vacuum filtration using filter paper to obtain a white solid content. Then, the obtained white solid content was washed with ethyl acetate and dried to obtain 7.6 g of a white powder.

A portion of the thus obtained powder was sampled, and subjected to a liquid chromatographic analysis (LC analysis: LC measurement). The result showed that the obtained white powder gave a single peak (a single product was obtained). Note that the result of the liquid chromatographic analysis showed that the raw material compound did not remain at all. In addition, to identify the structure of the compound of the thus obtained crystals, NMR measurement and LC measurement were conducted. The obtained compound was identified to be a compound (acid anhydride; molecular weight: 384.38) represented by the following general formula (6):

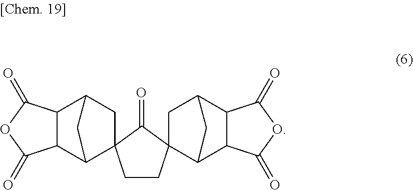

Note that the percentage yield of the thus obtained compound (acid anhydride) based on the theoretical amount of the product calculated from the initial amount of the raw material compound used was determined to be 94%.

Note that the obtained product was white, and no color development was detected under visual observation. In addition, a 5% by mass solution was prepared by dissolving the obtained product in N, N-dimethylacetamide. Then, the light transmittance at 400 nm was measured by using the solution as a measurement sample and a UV-Vis measuring apparatus (trade name: “UV-2550”) manufactured by Shimadzu Corporation as a measuring apparatus. The measured light transmittance at 400 nm was 98.2%. Table 1 shows the obtained results.

### Example 2

A product (7.4 g) was obtained in the same manner as in Example 1, except that 0.16 g of trifluoromethanesulfonic acid (TfOH, boiling point: 162° C.) ([amount of moles of raw material compound]: [amount of moles of functional groups (sulfonic acid) in catalyst)]=1:0.05) was used as a homogeneous acid catalyst instead of 0.38 g of tetrafluoroethanesulfonic acid, and that the time (heating time) at which the heating was stopped was changed from 4 hours to 6 hours in the step (i). Note that the heating time was determined based on the time passed by the time methyl acetate ceased to be distill off. In addition, to determine the acid strength of trifluoromethanesulfonic acid, the “acid dissociation constant (pKa)” was calculated by employing the above-described method for calculating an “acid dissociation constant (pKa)” according to the present invention. The calculated acid dissociation constant (pKa) was −9.0. In addition, the amount (total amount) of methyl acetate distilled off by the time 6 hours had passed since the removal by distillation was started was 5.1 g.

The thus obtained product was subjected to NMR measurement and LC measurement, and the product was identified to be a compound (acid anhydride) represented by the general formula (6). Note that the light transmittance at 400 nm was measured by using the obtained product in the same manner as in Example 1. The measured light transmittance at 400 nm was 98.4%. Table 1 shows the obtained results.

### Comparative Example 1

A product (6.4 g) was obtained in the same manner as in Example 1, except that 0.40 g of p-toluenesulfonic acid (p-TsOH, boiling point: 140° C.) ([amount of moles of raw material compound]:[amount of moles of functional groups (sulfonic acid) in catalyst)]=1:0.1) was used as a homogeneous acid catalyst instead of 0.38 g of tetrafluoroethanesulfonic acid, and that the time (heating time) at which the heating was stopped in the step (i) was changed from 4 hours to 48 hours. Note that the heating time was determined based on the time passed by the time methyl acetate ceased to be distilled off. In addition, to determine the acid strength of p-toluenesulfonic acid, the “acid dissociation constant (pKa)” was calculated by employing the above-described method for calculating an “acid dissociation constant (pKa)” according to the present invention. The calculated acid dissociation constant (pKa) was −0.6. In addition, the amount (total amount) of methyl acetate distilled off by the time 48 hours had passed since the removal by distillation was started was 5.1 g.

Under visual observation, the thus obtained product was found to be colored in gray, and it was not possible to obtain crystals in which color development was sufficiently suppressed, when p-toluenesulfonic acid was used as the acid catalyst. Note that the obtained product was subjected to NMR measurement and LC measurement, and the product was identified to be the compound (acid anhydride) represented by the general formula (6). Note that the light transmittance at 400 nm was measured by using the obtained product in the same manner as in Example 1. The measured light transmittance at 400 nm was 93.8%. Table 1 shows the obtained results.

### Comparative Example 2

A product (7.4 g) was obtained in the same manner as in Comparative Example 1, except that 0.2 g of sulfuric acid (H₂SO₄, boiling point: 290° C.) ([raw material compound (mol)]:[catalyst (mol)]=1:0.1) was used as a homogeneous acid catalyst instead of p-toluenesulfonic acid (p-TsOH), and that the time (heating time) at which the heating was stopped in the step (i) was changed from 48 hours to 20 hours. Note that the heating time was determined based on the time passed by the time methyl acetate ceased to be distilled off. In addition, to determine the acid strength of sulfuric acid, the “acid dissociation constant (pKa)” was calculated by employing the above-described method for calculating an “acid dissociation constant (pKa)” according to the present invention. The calculated acid dissociation constant (pKa) was −6.2. In addition, the amount (total amount) of methyl acetate distilled off by the time 20 hours had passed since the removal by distillation was started was 5.7 g.

Under visual observation, the thus obtained product was found to be colored in gray, and it was not possible to obtain crystals in which color development was sufficiently suppressed when sulfuric acid was used as the acid catalyst. Note that the obtained product was subjected to NMR measurement and LC measurement, and the product was identified to be the compound (acid anhydride) represented by the general formula (6). In addition, the light transmittance at 400 nm was measured by using the obtained product in the same manner as in Example 1. The light transmittance at 400 nm was 87.5%. Table 1 shows the obtained results.

TABLE 1

| | Homogeneous acid catalyst | | | | | | | | |
|---|---|---|---|---|---|---|---|---|---|
| | | Acid strength | | | | | | | |
| | | (pKa calculated | | | | | | | |
| | | based on | | | | Reaction | | | |
| | | quantum | Boiling | Mole ratio to | | time | | | |
| | | chemistry | point | raw material compound | Reaction | (heating | Color | Light | Yield of |
| | Species | calculation) | (° C.) | ([raw material]:[catalyst]) | temperature | time) | development | transmittance | product |
| Example 1 | HCF₂CF₂SO₃H | pKa = −9.5 | 210 | 1:0.1 | 118° C. | 4 h | None | 98.2% | 94% |
| Example 2 | TfOH | pKa = −9.0 | 162 | 1:0.05 | 118° C. | 6 h | None | 98.4% | 91% |
| Comp. Ex. 1 | p-TsOH | pKa = 0.6 | 140 | 1:0.1 | 118° C. | 48 h | Occurred | 93.8% | 80% |
| | | | | | | | (gray) | | |
| Comp. Ex. 2 | H₂SO₄ | pKa = −6.2 | 290 | 1:0.1 | 118° C. | 20 h | Occurred | 87.5% | 93% |
| | | | | | | | (gray) | | |

As is apparent from the results shown in Table 1, it was found that, in the cases (Examples 1 and 2) where the homogeneous acid catalysts were used which had acid dissociation constants (pKa) of −6.5 or lower and boiling points of 100° C. or higher, the acid dissociation constants (pKa) being determined by the quantum chemistry calculation (density functional method), crystals of the target compound (tetracarboxylic anhydride) in which color development was sufficiently suppressed were successfully produced efficiently. On the other hand, it was found that the obtained product was colored in gray under visual observation in each of the cases (Comparative Examples 1 and 2) where p-toluenesulfonic acid (acid strength: pKa=−0.6, boiling point: 140° C.) or sulfuric acid (acid strength: pKa=−6.2, boiling point: 290° C.) was used as the acid catalyst, although the mole ratios of the acid catalysts to the raw material compound were the same as that in Example 1, and it was not possible to sufficiently suppress the color development in the reaction. Moreover, a comparison between Example 1 and Comparative Examples 1 to 2, between which the mole ratios of the acid catalyst to the raw material compound were the same, showed that the reaction rate was improved by 5 times or more in the case (Example 1) where tetrafluoroethanesulfonic acid, which had an acid dissociation constant (pKa) of −6.5 or lower and a boiling point of 100° C. or higher, the acid dissociation constant (pKa) being determined by the quantum chemistry calculation, was used as the homogeneous acid catalyst in comparison with each of the cases (Comparative Examples 1 and 2) where p-toluenesulfonic acid (acid strength: pKa=−0.6, boiling point: 140° C.) or sulfuric acid (acid strength: pKa=−6.2, boiling point: 290° C.) was used as the homogeneous acid catalyst. In addition, it was found that the reaction rate was sufficiently improved in the case (Example 2) where trifluoromethanesulfonic acid, which had an acid dissociation constant (pKa) of −6.5 or lower and which had a boiling point of 100° C. or higher, the acid dissociation constant (pKa) being determined by the quantum chemistry calculation, was used as the homogeneous acid catalyst in comparison with each of the cases (Comparative Examples 1 and 2) where p-toluenesulfonic acid (acid strength: pKa=−0.6, boiling point: 140° C.) or sulfuric acid (acid strength: pKa=−6.2, boiling point: 290° C.) was used as the acid catalyst, although the ratio (mole ratio) of the acid catalyst used was smaller in Example 2. These results have shown that the present invention eventually makes it possible to reduce the amount of acetic acid removed by distillation, and provides a method sufficiently advantageous in terms of economical efficiency. In addition, also from the fact that the compounds obtained by the method for producing a carboxylic anhydride of the present invention (Examples 1 and 2) had light transmittances of 98.2% or higher, it was found that the color development in the crystals was sufficiently suppressed, and it has been found that the method for producing a carboxylic anhydride of the present invention (Examples 1 and 2) is especially useful as a method for producing a monomer (colorless material) used for producing a polyimide having a sufficiently high transparency, and the like.

From the results of Examples and Comparative Examples, it has been found that the use of the homogeneous acid catalyst which has such a sufficiently high acid strength that the pKa determined by the quantum chemistry calculation (the DFT calculation using the software manufactured by GAUSSIAN) based on the density functional method is −6.5 or lower and which has a boiling point of 100° C. or higher makes it possible to sufficiently suppress the color development from the original color of crystals.

From the above-described results, it has been found that the method for producing a carboxylic anhydride of the present invention (Examples 1 and 2) makes it possible to efficiently produce a carboxylic anhydride in which color development is sufficiently suppressed from the original color of crystals, although a homogeneous acid catalyst is used. In addition, it is also clear that, because of the use of the homogeneous acid catalyst, the method for producing a carboxylic anhydride of the present invention (Examples 1 and 2) makes it possible to produce the carboxylic anhydride more easily by further simplifying the steps in comparison with the case where a heterogeneous catalyst is used.

### INDUSTRIAL APPLICABILITY

As described above, according to the present invention, it is possible to provide a method for producing a carboxylic anhydride, the method being capable of efficiently producing a carboxylic anhydride in which color development was sufficiently suppressed from the original color of crystals, although a homogeneous catalyst is used.

Accordingly, the method for producing a carboxylic anhydride of the present invention is especially useful as a method for producing carboxylic anhydrides for use as raw materials for polyimides, polyesters, polyamides, and the like, curing agents for thermosetting resins, and the like, etc.

1. A method for producing a carboxylic anhydride, the method comprising
heating a raw material compound represented by the following general formula (1): 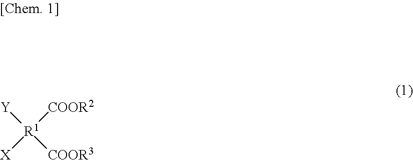
[in the formula (1),
R¹ is a tetravalent organic group having at least two carbon atoms adjacent to each other, groups represented by the formulae: —COOR² and —COOR³ are bonded to one and the other of the two adjacent carbon atoms, respectively,
R² and R³, which may be the same or different, each represent one selected from the group consisting of a hydrogen atom, alkyl groups having 1 to 10 carbon atoms, cycloalkyl groups having 3 to 10 carbon atoms, alkenyl groups having 2 to 10 carbon atoms, aryl groups having 6 to 20 carbon atoms, and aralkyl groups having 7 to 20 carbon atoms,
X represents one selected from the group consisting of a hydrogen atom, alkyl groups having 1 to 10 carbon atoms, alkenyl groups having 2 to 10 carbon atoms, and groups represented by the formula: —COOR⁴ (R⁴ has the same meaning as that of R², and may be the same as R² or different from R²), and
Y represents one selected from the group consisting of a hydrogen atom, alkyl groups having 1 to 10 carbon atoms, alkenyl groups having 2 to 10 carbon atoms, and groups represented by the formula: —COOR⁵ (R⁵ has the same meaning as that of R², and may be the same as R² or different from R²)]
in a carboxylic acid having 1 to 5 carbon atoms with a catalyst being used, to thereby obtain the carboxylic anhydride, wherein
the catalyst is a homogeneous acid catalyst having an acid dissociation constant (pKa) of −6.5 or lower and a boiling point of 100° C. or higher, the acid dissociation constant (pKa) being determined by a quantum chemistry calculation based on a density functional method.

2. The method for producing a carboxylic anhydride according to claim 1, wherein
the homogeneous acid catalyst is at least one selected from the group consisting of trifluoromethanesulfonic acid, tetrafluoroethanesulfonic acid, pentafluoroethanesulfonic acid, heptafluoropropanesulfonic acid, heptafluoroisopropanesulfonic acid, nonafluorobutanesulfonic acid, heptafluorodecanesulfonic acid, bis(nonafluorobutanesulfonyl)imide, N,N-bis(trifluoromethanesulfonyl)imide, and chlorodifluoroacetic acid.

3. The method for producing a carboxylic anhydride according to claim 1, wherein
the raw material compound is a spiro compound represented by the following general formula (2): 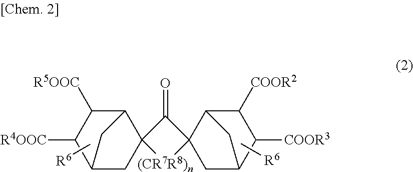
[in the formula (2), R², R³, R⁴, and R⁵ have the same meanings as those of R², R³, R⁴, and R⁵ described for the general formula (1), R⁶s, R⁷, and R⁸, which may be the same or different, each represent one selected from the group consisting of a hydrogen atom, alkyl groups having 1 to 10 carbon atoms, and a fluorine atom, and n represents an integer of 0 to 12].

4. The method for producing a carboxylic anhydride according to claim 2, wherein
the raw material compound is a spiro compound represented by the following general formula (2): 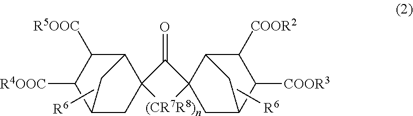
[in the formula (2), R², R³, R⁴, and R⁵ have the same meanings as those of R², R³, R⁴, and R⁵ described for the general formula (1), R⁶s, R⁷, and R⁸, which may be the same or different, each represent one selected from the group consisting of a hydrogen atom, alkyl groups having 1 to 10 carbon atoms, and a fluorine atom, and n represents an integer of 0 to 12].
